(19) United States
(12) Patent Application Publication
(10) Pub. No.: US 2022/0387283 A1
(43) Pub. Date: Dec. 8, 2022

(54) HAIR GROWTH AGENT

(71) Applicants: ADJUVANT HOLDINGS CO., LTD., Kobe-shi, Hyogo (JP); RIKEN, Wako-shi, Saitama (JP)

(72) Inventors: Sota Nakamura, Kobe-shi (JP); Hideki Takahashi, Kobe-shi (JP); Yukimi Nakaike, Kobe-shi (JP); Takahiro Nagaya, Kobe-shi (JP); Yuki Kanazawa, Kobe-shi (JP); Takashi Tsuji, Wako-shi, Saitama (JP); Miho Ogawa, Kobe-shi, Hyogo (JP)

(73) Assignees: ADJUVANT HOLDINGS CO., LTD., Kobe-shi, Hyogo (JP); RIKEN, Wako-shi, Saitama (JP)

(21) Appl. No.: 17/769,990

(22) PCT Filed: Sep. 23, 2020
(86) PCT No.: PCT/JP2020/035859

§ 371 Date: Apr. 18, 2022

(30) Foreign Application Priority Data
Oct. 18, 2019 (JP) 2019-190689

(51) Int. Cl. A61K 8/64; A61K 38/05; A61K 8/49; A61Q 7/00

(52) U.S. Cl. CPC A61K 8/64; A61K 38/05; A61K 8/4913; A61Q 7/00

(57) ABSTRACT

To provide a hair growth agent which is a topical agent that exhibits effect in terms of causing increase in hair shaft diameter and improving maximum hair shaft length and improving hair shaft elongation rate and new hair growth and increasing expression of genes contributing to hair growth in dermal papilla cells and promoting hair shaft growth at head hair, eyelashes, and/or eyebrows, a hair growth agent which is a topical agent is made to contain active ingredients in the form of palmitoyl dipeptide-5 diaminobutyloyl hydroxythreonine and palmitoyl dipeptide-5 diaminohydroxybutyrate.

### TECHNICAL FIELD

The present invention relates to a hair growth agent. More particularly, it relates to a hair growth agent that is a topical agent which contains palmitoyl dipeptide-5 diaminobutyloyl hydroxythreonine and palmitoyl dipeptide-5 diaminohydroxybutyrate.

### BACKGROUND ART

There has been increasing demand for hair growth agents and other such topical agents that will improve hair type and/or hair quality and hair growth effect in mammals including humans. To improve hair type and/or hair quality and hair growth effect, active ingredients which contribute to regulation of the hair cycle, i.e., the hair life cycle, have been proposed and are in the process of coming onto the market in the form of hair growth agents.

For example, use of minoxidil as an active ingredient in a hair growth agent has been proposed (see Patent Reference Nos. 1 through 3 and so forth), and hair growth agents employing minoxidil as active ingredient have undergone clinical trials in humans and are on the market. However, for reasons such as the fact that the pharmaceutical use thereof within Japan is limited to male alopecia prematura, it has not adequately satisfied the broad needs of consumers who desire hair growth effect and hair type and/or hair quality improvement effect.

Furthermore, use of chiro-inositol as active ingredient in hair growth agent has been proposed (see Patent Reference No. 4). However, as the hair growth effect of the hair growth agent which is a topical agent and which contains chiro-inositol that is described at Patent Reference No. 4 has only been demonstrated for non-insulin-resistant subjects, the subjects to whom it may be administered are limited. This being the case, it has not adequately satisfied the broad needs of consumers who desire hair growth effect and hair type and/or hair quality improvement effect.

Palmitoyl dipeptide-5 diaminobutyloyl hydroxythreonine and palmitoyl dipeptide-5 diaminohydroxybutyrate are known as raw materials for cosmetics (see Patent Reference No. 5). However, there are no reports related to a hair growth effect of palmitoyl dipeptide-5 diaminobutyloyl hydroxythreonine and palmitoyl dipeptide-5 diaminohydroxybutyrate.

### PRIOR ART REFERENCES

### Patent References

Patent Reference No. 1: Specification of U.S. Pat. No. 4,139,619
Patent Reference No. 2: Japanese Patent Application Publication Kokai No. S63[1988]-150211
Patent Reference No. 3: Japanese Patent Application Publication Kokai No. S63[1988]-145217
Patent Reference No. 4: International Patent Application Publication No. 2017/188393
Patent Reference No. 5: Japanese Patent No. 5028474

### SUMMARY OF INVENTION

### Problem to be Solved by Invention

It is an object of the present invention to provide a hair growth agent that possesses excellent hair growth action.

### Means for Solving Problem

As a result of intensive and repeated research for the purpose of solving the foregoing problems, the present inventor(s) discovered that use of palmitoyl dipeptide-5 diaminobutyloyl hydroxythreonine and palmitoyl dipeptide-5 diaminohydroxybutyrate as active ingredients permitted attainment of excellent hair growth activity, which culminated in the present invention.

A first means in accordance with the present invention for solving the foregoing problems is a hair growth agent which is a topical agent that contains palmitoyl dipeptide-5 diaminobutyloyl hydroxythreonine and palmitoyl dipeptide-5 diaminohydroxybutyrate.

A second means in accordance with the present invention for solving the foregoing problems is the hair growth agent of the first means in accordance with the present invention wherein the palmitoyl dipeptide-5 diaminobutyloyl hydroxythreonine and the palmitoyl dipeptide-5 diaminohydroxybutyrate are present therein in an amount that is 0.001 wt % to 20 wt % of the entirety.

A third means in accordance with the present invention for solving the foregoing problems is the hair growth agent of the first means or the second means in accordance with the present invention wherein the palmitoyl dipeptide-5 diaminobutyloyl hydroxythreonine and the palmitoyl dipeptide-5 diaminohydroxybutyrate are present therein in an amount that is 0.005 wt % to 10 wt % of the entirety.

A fourth means in accordance with the present invention for solving the foregoing problems is the hair growth agent of any one among the first through third means in accordance with the present invention wherein the palmitoyl dipeptide-5 diaminobutyloyl hydroxythreonine and the palmitoyl dipeptide-5 diaminohydroxybutyrate are present therein such that a ratio thereof when expressed as a ratio of percents by mass (the palmitoyl dipeptide-5 diaminobutyloyl hydroxythreonine/the palmitoyl dipeptide-5 diaminohydroxybutyrate) is 99/1 to 1/99.

A fifth means in accordance with the present invention for solving the foregoing problems is the hair growth agent of any one among the first through fourth means in accordance with the present invention for use in causing new hair growth or hair shaft growth promotion.

A sixth means in accordance with the present invention for solving the foregoing problems is the hair growth agent of any one among the first through fifth means in accordance with the present invention used for causing improvement in hair shaft elongation rate.

A seventh means in accordance with the present invention for solving the foregoing problems is the hair growth agent of any one among the first through fifth means in accordance with the present invention used for causing improvement in maximum hair shaft length.

An eighth means in accordance with the present invention for solving the foregoing problems is the hair growth agent of any one among the first through fifth means in accordance with the present invention used for causing increase in hair shaft diameter.

A ninth means in accordance with the present invention for solving the foregoing problems is the hair growth agent of any one among the first through fifth means in accordance with the present invention used for causing increase in number of hairs.

A tenth means in accordance with the present invention for solving the foregoing problems is the hair growth agent of any one among the first through ninth means in accordance with the present invention in liquid solution form.

An eleventh means in accordance with the present invention for solving the foregoing problems is the hair growth agent of any one among the first through tenth means in accordance with the present invention for use on head hair, eyelashes, and/or eyebrows.

A twelfth means in accordance with the present invention for solving the foregoing problems is a hair growth method comprising administering the hair growth agent of any one among the first through eleventh means in accordance with the present invention to a subject.

Another means in accordance with the present invention for solving the foregoing problems is a scalp care agent which is a topical agent that contains palmitoyl dipeptide-5 diaminobutyloyl hydroxythreonine and palmitoyl dipeptide-5 diaminohydroxybutyrate.

Another means in accordance with the present invention for solving the foregoing problems is a scalp symptom improvement method comprising administering a scalp care agent which is a topical agent that contains palmitoyl dipeptide-5 diaminobutyloyl hydroxythreonine and palmitoyl dipeptide-5 diaminohydroxybutyrate to a subject.

### Benefit of Invention

By causing palmitoyl dipeptide-5 diaminobutyloyl hydroxythreonine and palmitoyl dipeptide-5 diaminohydroxybutyrate to be active ingredients in a hair growth agent which is a topical agent, means in accordance with the present invention make it is possible to provide an excellent hair growth agent and scalp care agent that exhibit scalp care effect as well as effect in terms of causing increase in hair shaft diameter and improving maximum hair shaft length and improving hair shaft elongation rate and hair shaft growth promotion at head hair, eyelashes, and/or eyebrows.

### BRIEF DESCRIPTION OF DRAWINGS

FIG. 1 are plots showing change in hair shaft length, following application of 60% aqueous ethanol solution, at location where drug was applied in mice. The vertical axis shows hair shaft length (mm); the horizontal axis shows number of days. Note that the first hair cycle shows reference data for which a non-drug-containing 60% aqueous ethanol solution was applied.

FIG. 2 are plots showing change in hair shaft length, following application of 60% aqueous ethanol solution that contained minoxidil (5%), at location where drug was applied in mice. The vertical axis shows hair shaft length (mm); the horizontal axis shows number of days. Note that the first hair cycle shows reference data for which a non-drug-containing 60% aqueous ethanol solution was applied.

FIG. 3 are plots showing change in hair shaft length, following application of 60% aqueous ethanol solution that contained minoxidil (3%), at location where drug was applied in mice. The vertical axis shows hair shaft length (mm); the horizontal axis shows number of days. Note that the first hair cycle shows reference data for which a non-drug-containing 60% aqueous ethanol solution was applied.

FIG. 4 are plots showing change in hair shaft length, following application of 60% aqueous ethanol solution that contained minoxidil (1%), at location where drug was applied in mice. The vertical axis shows hair shaft length (mm); the horizontal axis shows number of days. Note that the first hair cycle shows reference data for which a non-drug-containing 60% aqueous ethanol solution was applied.

FIG. 5 are plots showing change in hair shaft length, following application of 60% aqueous ethanol solution that contained palmitoyl dipeptide-5 diaminobutyloyl hydroxythreonine and palmitoyl dipeptide-5 diaminohydroxybutyrate (3%), at location where drug was applied in mice. The vertical axis shows hair shaft length (mm); the horizontal axis shows number of days. Note that the first hair cycle shows reference data for which a non-drug-containing 60% aqueous ethanol solution was applied.

FIG. 6 are plots showing change in hair shaft length, following application of 60% aqueous ethanol solution that contained palmitoyl dipeptide-5 diaminobutyloyl hydroxythreonine and palmitoyl dipeptide-5 diaminohydroxybutyrate (1%), at location where drug was applied in mice. The vertical axis shows hair shaft length (mm); the horizontal axis shows number of days. Note that the first hair cycle shows reference data for which a non-drug-containing 60% aqueous ethanol solution was applied.

FIG. 7 shows evaluation of mitogenic activity caused by a mixture of palmitoyl dipeptide-5 diaminobutyloyl hydroxythreonine and palmitoyl dipeptide-5 diaminohydroxybutyrate. Cells for which uptake of BrdU could be confirmed are indicated by upwardly directed arrowheads.

FIG. 8 shows evaluation of mitogenic activity caused by a mixture of palmitoyl dipeptide-5 diaminobutyloyl hydroxythreonine and palmitoyl dipeptide-5 diaminohydroxybutyrate.

FIG. 9 shows results of measurement of hair shaft diameter following application of drug.

FIG. 10 contains graphs showing change in amount of genetic expression as a result of stimulation for 24 hours with a mixture of palmitoyl dipeptide-5 diaminobutyloyl hydroxythreonine and palmitoyl dipeptide-5 diaminohydroxybutyrate in human dermal papilla cells. (a) at FIG. 10 shows change in amount of genetic expression of FGF-7; (b) at FIG. 10 shows change in amount of genetic expression of VEGF.

FIG. 11 contains graphs showing change in amount of genetic expression as a result of stimulation for 72 hours with a mixture of palmitoyl dipeptide-5 diaminobutyloyl hydroxythreonine and palmitoyl dipeptide-5 diaminohydroxybutyrate in human dermal papilla cells. (a) at FIG. 11 shows change in amount of genetic expression of FGF-7; (b) at FIG. 11 shows change in amount of genetic expression of VEGF.

### EMBODIMENTS FOR CARRYING OUT INVENTION

Embodiments for carrying out the present invention are described below. Note that the present invention is not limited to these examples alone, it being of course possible to make any number of changes thereto without departing from the gist of the present invention.

The active ingredients of a hair growth agent and a scalp care agent that are topical agents associated with the present invention comprise palmitoyl dipeptide-5 diaminobutyloyl hydroxythreonine (Palm-Lys-Val-Dab-Thr-OH) and palmitoyl dipeptide-5 diaminohydroxybutyrate (Palm-Lys-Val-Dab-OH).

Concentration of the mixture of palmitoyl dipeptide-5 diaminobutyloyl hydroxythreonine and palmitoyl dipeptide-5 diaminohydroxybutyrate constituting active ingredients in a hair growth agent and scalp care agent in accordance with the present invention is 0.001 wt % to 20 wt % of the entirety of the entire hair growth agent and scalp care agent. More specifically, it is 0.005 wt % to 10 wt %.

A hair growth agent which contains palmitoyl dipeptide-5 diaminobutyloyl hydroxythreonine and palmitoyl dipeptide-5 diaminohydroxybutyrate in accordance with the present invention is such that the ratio thereof when expressed as a ratio of percents by mass (palmitoyl dipeptide-5 diaminobutyloyl hydroxythreonine/palmitoyl dipeptide-5 diaminohydroxybutyrate) is 99/1 to 1/99.

While hair growth agents and scalp care agents in accordance with the present invention may be used in the form of pharmaceutical preparations of any of a wide variety of modes such as ointments, poultices, liniments, lotions, liquids for topical use, dusting powders, creams, gels, emulsions, hair tonics, hair sprays, and so forth as cosmetics including cosmetics for the scalp and cosmetics for the eyelashes and/or eyebrows, head hair, quasi-pharmaceutical agents, pharmaceutical agents, and so forth, there is no limitation with respect thereto.

Furthermore, to the extent that it does not interfere with the hair growth effect and scalp care effect of the present invention, additives and/or other such components, presence of which would ordinarily be permitted in cosmetics including cosmetics for the scalp and cosmetics for the eyelashes and/or eyebrows, head hair, quasi-pharmaceutical agents, pharmaceutical agents, and so forth, may be additionally blended therein. As such additives and/or other such components, while excipients, stabilizers, corrigents, vehicle, dispersants, diluents, anionic surface active agents, amphoteric surface active agents, nonionic surface active agents, cationic surface active agents, anionic polymers, nonionic polymers, ethylene oxide-propylene oxide block copolymer, alcohols, emulsifiers, percutaneous absorption promoters, pH adjustors, preservatives, colorants, lipids, mineral oils, and other such oily components, moisturizing agents, thickeners, polymers, film-forming agents, ultraviolet light absorbers, cell activators, moisturizing agents, inorganic salts, functional beads and capsules, silicones, metal chelating agents, antioxidants, antiseptic agents, fresheners, deodorants, pigments, dyes, fragrances, sugars, amino acids, vitamins, organic acids, organic amines, plant extracts, clay minerals, various polymers, and other such viscosity modifiers, and so forth may be cited as examples, there is no limitation with respect thereto.

Hair growth agents and scalp care agents in accordance with the present invention may contain known components having new hair growth effect, hair growth effect, hair tonic effect, and/or the like.

Administration dosage of active ingredients per dose of a hair growth agent and scalp care agent of a means in accordance with the present invention may be adjusted so as to cause effect(s) of the hair growth agent and scalp care agent in accordance with the present invention to be exhibited. In addition, such administration dosage might for example be 0.005 mg to 200 mg, might more specifically be 0.05 mg to 100 mg, and might still more specifically be 0.5 mg to 10 mg.

So as to cause effect(s) of the hair growth agent and scalp care agent in accordance with the present invention to be exhibited, the number of administrations of a hair growth agent and scalp care agent in accordance with the present invention might be one administration or might be multiple administrations. In addition, the number of administrations of a hair growth agent and scalp care agent in accordance with the present invention might for example be 1 to 6 times per day. In addition, more specifically this might be 1 to 3 times per day, and still more specifically this might be 1 to 2 times per day.

Hair growth agents and scalp care agents in accordance with the present invention relate to hair shaft growth promotion, new hair growth, and hair loss prevention, and preferably relate to hair shaft growth promotion and new hair growth.

In the present specification, the term “hair shaft growth promotion” means improving hair shaft elongation rate, improving maximum hair shaft length, and/or increasing hair shaft diameter.

In the present specification, the term “new hair growth” means promoting growth of new hair and increasing number of hairs at follicle pores where new hair growth capability has been lowered or where new hair growth has stopped at a location where there are a small number of hairs or where there is no hair (no hair shaft extends to the exterior from the epidermis), and more specifically means shortening the telogen phase of the hair cycle and/or restarting a stopped hair cycle.

In the present specification, “to have hair shaft growth promotion effect” means acting in a way such as will be advantageous for promotion of hair shaft growth, and the quality by which hair shaft growth promotion effect is indicated is referred to as “hair shaft growth promotion activity”. Furthermore, “to have new hair growth effect” means acting in a way such as will be advantageous for new hair growth, and the quality by which new hair growth effect is indicated is referred to as “new hair growth promotion activity”.

In the present specification, the term “hair loss” means the phenomenon whereby the hair shaft comes free from the follicle pore, and more specifically means increase in inhibitory cytokines or the like which interfere with cell growth, and to cell death resulting therefrom. The quality by which hair loss prevention effect is indicated is referred to as “hair loss prevention activity”. Furthermore, “to have hair loss prevention effect,” which is a physiological phenomenon different from the qualities by which hair shaft growth promotion and/or new hair growth effect are indicated, means decreasing the number of hair shafts that come free from follicle pores as a result of reduction in or interference with inhibitory cytokines and suppression of cell death.

In the present specification, the term “scalp symptoms” means dandruff, roughness of the scalp, dryness of the scalp, erythema, itchiness, acne, and/or other such symptoms. In addition, in the present specification, the term “improvement of scalp symptoms” means improvement or suppression of dandruff, roughness of the scalp, dryness of the scalp, erythema, itchiness, acne, and/or the like.

A hair growth agent in accordance with the present invention may be used to improve hair shaft elongation rate and/or maximum hair shaft length. In addition, with respect to hair shaft elongation rate, as compared with hair shaft elongation rate pursuant to hair cycle reference data, it may for example cause a maximum improvement of on the order of 110%, more specifically it may cause improvement on the order of 25% to 110%, and still more specifically it may cause improvement on the order of 33% to 110%. Furthermore, with respect to maximum hair shaft length, as compared with maximum hair shaft length pursuant to hair cycle reference data, it may for example cause a maximum improvement of on the order of 49%, more specifically it may cause improvement on the order of 1% to 49%, and still more specifically it may cause improvement on the order of 2% to 49%.

A hair growth agent in accordance with the present invention may be used to increase hair shaft diameter.

A hair growth agent in accordance with the present invention may be used to promote growth of new hair and increase the number of hairs at follicle pores where new hair growth capability has been lowered or where new hair growth has stopped at a location where there are a small number of hairs or where there is no hair (no hair shaft extends to the exterior from the epidermis), and more specifically may be used to shorten the telogen phase of the hair cycle and/or restart a stopped hair cycle.

Hair growth agents and scalp care agents in accordance with the present invention may be used not only for humans but also for domesticated animals, animal pets, and/or other such animals. One aspect of the present invention provides a scalp symptom improvement method and a hair growth method that includes administration of a topical agent which contains palmitoyl dipeptide-5 diaminobutyloyl hydroxythreonine and palmitoyl dipeptide-5 diaminohydroxybutyrate to subject(s) which may include human(s), domesticated animal(s), animal pet(s), and/or other such animal(s).

### WORKING EXAMPLES

### Exemplary Test 1: Evaluation of Hair Growth Activity Caused by a Mixture of Palmitoyl Dipeptide-5 Diaminobutyloyl Hydroxythreonine and Palmitoyl Dipeptide-5 Diaminohydroxybutyrate

### 1. Materials and Methods

### (1) Experimental Animals

C57BL/6N mice (male) and Balb/c nu/nu mice (female) were purchased from Japan SLC, Inc. (Japan) and bred, and were thereafter made available for the following testing. Note that the testing and breeding of animals complied with pertinent laws, regulations, ordinances, and guidelines, and was performed with the approval of the Experimental Ethics Review Board of the Institute of Physical and Chemical Research.

### (2) Reagents

The following reagents were respectively prepared.

Comparative Example 1: 60% aqueous ethanol solution
Comparative Example 2: 5% minoxidil solution
Comparative Example 3: 3% minoxidil solution
Comparative Example 4: 1% minoxidil solution
Working Example 1: 3% solution of mixture of palmitoyl dipeptide-5 diaminobutyloyl hydroxythreonine and palmitoyl dipeptide-5 diaminohydroxybutyrate
Working Example 2: 1% solution of mixture of palmitoyl dipeptide-5 diaminobutyloyl hydroxythreonine and palmitoyl dipeptide-5 diaminohydroxybutyrate
(3) Preparation of Skin Samples Derived from Mouse Dorsal Body Hair Skin

To collect dorsal body hair skin in the form of anagen stage VI skin 12 to 14 days following depilation, C57BL/6N mice of age 7 to 8 weeks were depilated at locations where dorsal body hair skin was intended to be collected, and were bred for 12 to 14 days. The depilated C57BL/6N mice were thereafter euthanized by cervical dislocation, following which a suitable amount of dorsal body hair skin was collected from the locations at which dorsal body hair skin was intended to be collected.

The collected skin was immersed in DMEM culture medium (hereinafter “DMEM 10”) which contained 10 mM HEPES, 10% fetal bovine serum, and 1% penicillin/streptomycin solution. The collected dorsal body hair skin was grasped with bent-nose curved tweezers and was treated by immersion for 10 seconds in a sterilizing solution. Sterilization treatment was performed by carrying out treatment with 7% povidone iodine solution two times, treatment with PBS (−) three times, and treatment with DMEM 10 two times, in this order, with fresh solutions respectively being used each time. Following sterilization treatment, these were immersed in clean DMEM 10.

Following sterilization treatment, the dorsal body hair skin was cut into pieces and formed into blocks. The transparent connective tissue which adhered to the cutaneous muscle layer of the skin was excised therefrom using curved scissors, and hair groups were cut into rectangular strips in parallel fashion with respect to the direction of the wave of the hair. At this time, these were cut into blocks such that there were 6 rows of hair follicles along the long axis, adjustment having been carried out so that there were 5 rows of hair follicles along the short axis.

(4) Grafting of Skin Samples onto Balb/c Nu/Nu Mice

The skin samples derived from dorsal body hair skin that were prepared in accordance with the foregoing were grafted onto Balb/c nu/nu mice of age 4 to 6 weeks. Mice were anesthetized in the usual way using isoflurane gas. The dorsal area of the mice was then disinfected using 7% povidone iodine solution, following which the mice were made to assume a naturally recumbent posture. In addition, a Mani ophthalmic knife (Mani, Inc.; Japan) was used to pierce the skin at the dorsal area of the mice, the grafts which were formed extending from the epidermal layer of the skin to the subdermal layer.

The skin samples derived from dorsal body hair skin were inserted into the grafts formed thereat in such fashion as to cause the hair groups to be directed toward the body surface side of the grafts. Skin sample transplanted depth was adjusted so as to cause the top portion of the hair group to be in a state such that it was exposed at the top portion of the graft. To protect the grafts, Nurseban (registered trademark) (Sunplanet Co., Ltd.; Japan) and surgical tape (3M Japan Limited; Japan) were then used as protective tape to cover the grafts at which the skin samples derived from dorsal body hair skin had been transplanted.

The protective tape was removed 5 to 7 days following transplantation, and survival of the transplanted skin samples derived from dorsal body hair skin was determined by visual inspection or digital microscopy (Keyence Corporation; Japan), after which follow-up observation was carried out.

### (5) Application of Drug on Transplanted Skin Samples

For the first hair cycle, 60% aqueous ethanol solution was applied thereto as placebo. A micropipette was used to apply 25 μL of 60% ethanol respectively to the left and right dorsal regions of skin samples that survived in Balb/c nu/nu mice in which skin samples had been transplanted in accordance with the foregoing. A dryer was thereafter used to cause cool air to be directed thereat and rapidly dry the ethanol. This procedure was carried out in repetitive fashion four times at each the left and right dorsal regions of the mice.

For the second and subsequent hair cycles, solutions that were mixtures of palmitoyl dipeptide-5 diaminobutyloyl hydroxythreonine and palmitoyl dipeptide-5 diaminohydroxybutyrate of the respective concentrations were applied instead of 60% aqueous ethanol solution in accordance with the foregoing method to the Balb/c nu/nu mice in which the hair groups had been transplanted.

### (6) Histologic Analysis and Follow-Up Observation of New Hair Growth

Three regions were selected from the locations at which the skin samples were transplanted in Balb/c nu/nu mice, the situation with respect to new hair growth being determined and recorded for five hairs selected from each of these regions. Observation and recording was carried out by visual inspection and digital microscopy (Keyence Corporation; Japan).

### 2. Results

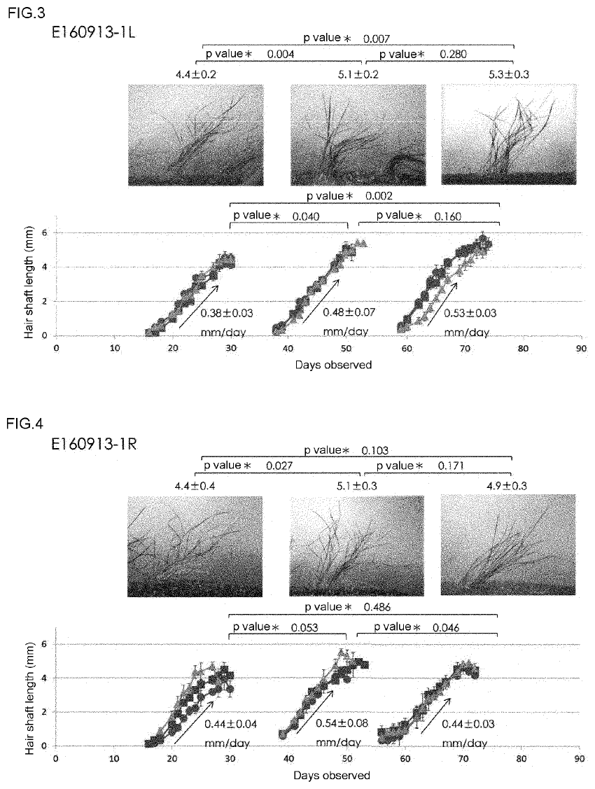

For each drug, hair shaft length was measured once every 2 to 4 days, the average of the hair shaft lengths at any given time being plotted as a single data point on a graph showing the change thereof with respect to time, similar plots being made for each of three mice. Results are shown in TABLES 1 to 6 and in FIGS. 1 to 6.

TABLE 1

| | | Data for situation | Data for situation |
|---|---|---|---|
| | Reference | in which drug | in which drug |
| | data | acted thereon | acted thereon |
| | (first hair | (second hair | (third hair |
| | cycle) | cycle) | cycle) |
| Hair shaft | 0.37 ± 0.04 | 0.41 ± 0.07 | 0.43 ± 0.04 |
| elongation | | | |
| rate (mm/day) | | | |
| Percent change | | 110.18% | 113.90% |
| relative to | | | |
| reference data | | | |
| Maximum hair | 4.6 ± 0.6 | 4.8 ± 0.4 | 4.7 ± 0.4 |
| shaft length (mm) | | | |
| Percent change | | 104.25% | 101.91% |
| relative to | | | |
| reference data | | | |

TABLE 2

| | | Data for situation | Data for situation |
|---|---|---|---|
| | Reference | in which drug | in which drug |
| | data | acted thereon | acted thereon |
| | (first hair | (second hair | (third hair |
| | cycle) | cycle) | cycle) |
| Hair shaft | 0.44 ± 0.04 | 0.51 ± 0.03* | 0.55 ± 0.07* |
| elongation | | | |
| rate (mm/day) | | | |
| Percent change | | 116.25% | 126.11% |
| relative to | | | |
| reference data | | | |
| Maximum hair | 5.2 ± 0.7 | 6.0 ± 0.1 | 5.8 ± 0.1 |
| shaft length (mm) | | | |
| Percent change | | 115.74% | 112.62% |
| relative to | | | |
| reference data | | | |

At TABLE 2, above,
*indicates p < 0.05, i.e., that the results are significant.

TABLE 3

| | | Data for situation | Data for situation |
|---|---|---|---|
| | Reference | in which drug | in which drug |
| | data | acted thereon | acted thereon |
| | (first hair | (second hair | (third hair |
| | cycle) | cycle) | cycle) |
| Hair shaft | 0.38 ± 0.03 | 0.48 ± 0.07* | 0.53 ± 0.03** |
| elongation | | | |
| rate (mm/day) | | | |
| Percent change | | 126.50% | 139.36% |
| relative to | | | |
| reference data | | | |
| Maximum hair | 4.4 ± 0.2 | 5.2 ± 0.2* | 5.3 ± 0.3 |
| shaft length (mm) | | | |
| Percent change | | 116.80% | 120.09% |
| relative to | | | |
| reference data | | | |

At TABLE 3, above,
*indicates p < 0.05 and
**indicates p < 0.01, i.e., that the results are significant.

TABLE 4

| | | Data for situation | Data for situation |
|---|---|---|---|
| | Reference | in which drug | in which drug |
| | data | acted thereon | acted thereon |
| | (first hair | (second hair | (third hair |
| | cycle) | cycle) | cycle) |
| Hair shaft | 0.44 ± 0.04 | 0.54 ± 0.08 | 0.44 ± 0.02 |
| elongation | | | |
| rate (mm/day) | | | |
| Percent change | | 123.85% | 100.24% |
| relative to | | | |
| reference data | | | |
| Maximum hair | 4.4 ± 0.4 | 5.2 ± 0.3** | 4.9 ± 0.4** |
| shaft length (mm) | | | |
| Percent change | | 118.07% | 111.10% |
| relative to | | | |
| reference data | | | |

At TABLE 4, above,
**indicates p < 0.01, i.e., that the results are significant.

TABLE 5

| | | Data for situation | Data for situation |
|---|---|---|---|
| | Reference | in which drug | in which drug |
| | data | acted thereon | acted thereon |
| | (first hair | (second hair | (third hair |
| | cycle) | cycle) | cycle) |
| Hair shaft | 0.44 ± 0.04 | 0.56 ± 0.03** | 0.50 ± 0.03* |
| elongation | | | |
| rate (mm/day) | | | |
| Percent change | | 128.76% | 115.46% |
| relative to | | | |
| reference data | | | |
| Maximum hair | 4.7 ± 0.6 | 5.3 ± 0.1 | 5.1 ± 0.1 |
| shaft length (mm) | | | |
| Percent change | | 112.34% | 108.69% |
| relative to | | | |
| reference data | | | |

At TABLE 5, above,
*indicates p < 0.05 and
**indicates p < 0.01, i.e., that the results are significant.

TABLE 6

| | | Data for situation | Data for situation |
|---|---|---|---|
| | Reference | in which drug | in which drug |
| | data | acted thereon | acted thereon |
| | (first hair | (second hair | (third hair |
| | cycle) | cycle) | cycle) |
| Hair shaft | 0.43 ± 0.03 | 0.41 ± 0.05 | 0.43 ± 0.04 |
| elongation | | | |
| rate (mm/day) | | | |
| Percent change | | 97.31% | 103.23% |
| relative to | | | |
| reference data | | | |
| Maximum hair | 4.0 ± 0.2 | 4.8 ± 0.3* | 4.6 ± 0.8 |
| shaft length (mm) | | | |
| Percent change | | 121.43% | 103.23% |
| relative to | | | |
| reference data | | | |

At TABLE 6, above,
*indicates p < 0.05, i.e., that the results are significant.

When 60% aqueous ethanol solution was applied to the locations at which the skin samples had been transplanted in mice, no hair growth activity was observed, there being no significant difference with respect to either hair shaft elongation rate or maximum hair shaft length as compared with reference data pertaining to the situation in which no solution was applied (see TABLE 1 and FIG. 1).

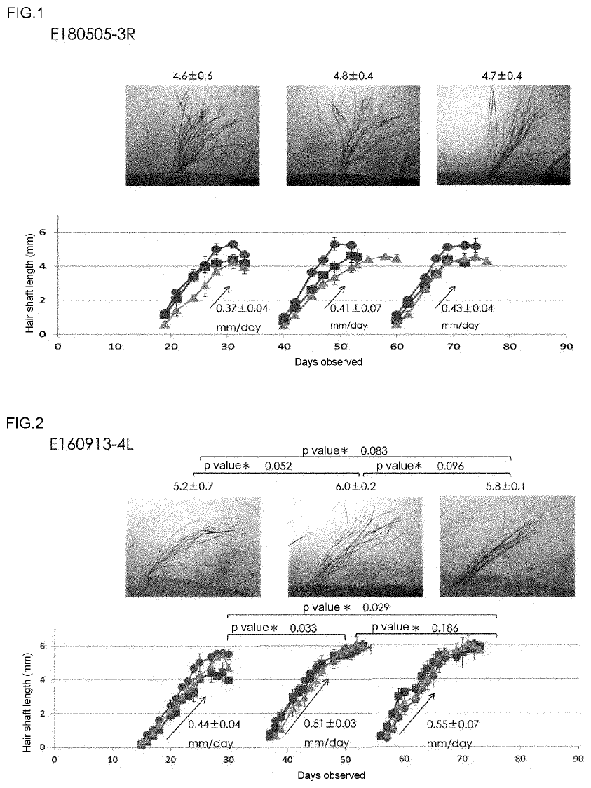

When a solution containing 5%, 3%, or 1% minoxidil was applied to the locations at which the skin samples had been transplanted in mice, hair growth activity was observed, there being significant improvement in both hair shaft elongation rate and maximum hair shaft length as compared with reference data (see TABLES 2 to 4 and FIGS. 2 to 4).

On the other hand, when a solution containing 3% of a mixture of palmitoyl dipeptide-5 diaminobutyloyl hydroxythreonine and palmitoyl dipeptide-5 diaminohydroxybutyrate was applied to the locations at which the skin samples had been transplanted in mice, there was significant improvement in hair shaft elongation rate as compared with reference data. Furthermore, with respect to maximum hair shaft length, while no significant difference was observed there was improvement (see TABLE 5 and FIG. 5). Based on these results, the solution that contained 3% of a mixture of palmitoyl dipeptide-5 diaminobutyloyl hydroxythreonine and palmitoyl dipeptide-5 diaminohydroxybutyrate was found to exhibit hair growth activity.

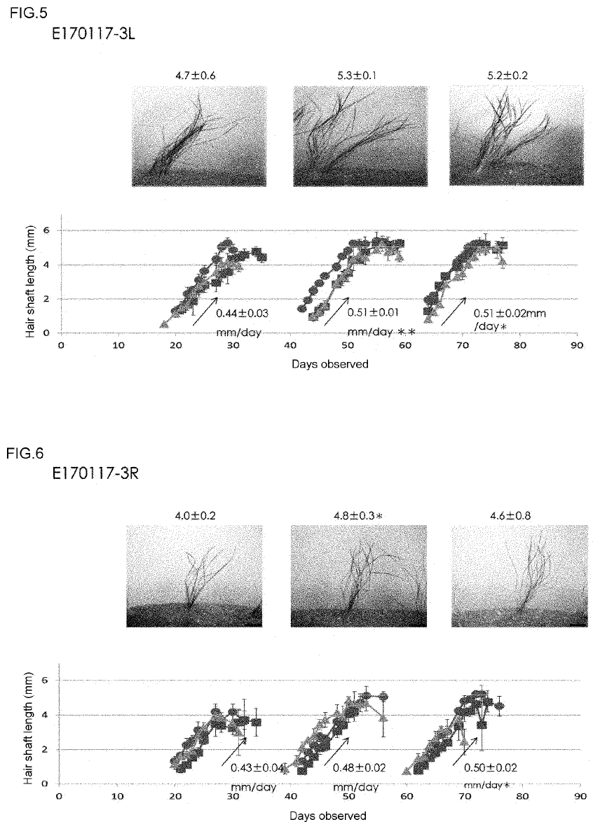

On the other hand, when a solution containing 1% of a mixture of palmitoyl dipeptide-5 diaminobutyloyl hydroxythreonine and palmitoyl dipeptide-5 diaminohydroxybutyrate was applied to the locations at which the skin samples had been transplanted in mice, no significant difference was observed with respect to hair shaft elongation rate at the second hair cycle, and while no significant difference was observed there was improvement with respect to maximum hair shaft length at the third hair cycle, as compared with reference data (see TABLE 6 and FIG. 6). Based on these results, there was a significant trend with respect to hair growth activity for the solution that contained 1% of a mixture of palmitoyl dipeptide-5 diaminobutyloyl hydroxythreonine and palmitoyl dipeptide-5 diaminohydroxybutyrate.

This therefore suggested that the lower limit of the range of concentrations for which palmitoyl dipeptide-5 diaminobutyloyl hydroxythreonine and palmitoyl dipeptide-5 diaminohydroxybutyrate is effective in producing hair growth activity when expressed in terms of the content of a mixture of palmitoyl dipeptide-5 diaminobutyloyl hydroxythreonine and palmitoyl dipeptide-5 diaminohydroxybutyrate is between 1% and 3%.

### Exemplary Test 2: Evaluation of Mitogenic Activity Caused by a Mixture of Palmitoyl Dipeptide-5 Diaminobutyloyl Hydroxythreonine and Palmitoyl Dipeptide-5 Diaminohydroxybutyrate

### 1. Materials and Methods

### (1) Experimental Animals

Dorsal body hair skin was collected from C57BL/6N mice (Japan SLC, Inc.; Japan) of age 6 to 8 weeks. Testing and breeding of animals complied with pertinent laws, regulations, ordinances, and guidelines, and was performed with the approval of the Experimental Ethics Review Board of the Institute of Physical and Chemical Research.

### (2) Drugs

The following drugs were respectively prepared.

Working Example 1: 3% solution of mixture of palmitoyl dipeptide-5 diaminobutyloyl hydroxythreonine and palmitoyl dipeptide-5 diaminohydroxybutyrate
Working Example 2: 1% solution of mixture of palmitoyl dipeptide-5 diaminobutyloyl hydroxythreonine and palmitoyl dipeptide-5 diaminohydroxybutyrate
Comparative Example 1: 60% aqueous ethanol solution

### Comparative Example 5: RiUP X5 Plus (Taisho Pharmaceutical Co., Ltd.; Japan)

### (3) Application of Drug

C57BL/6N mice were depilated of body hair at the dorsal region. A micropipette was used to apply 25 μL of the drug to the left and right dorsal regions of the C57BL/6N mice. A dryer was thereafter used to cause cool air to be directed at the location where this was applied and rapidly dry the drug. This procedure was carried out in repetitive fashion four times at each the left and right dorsal regions. This drug application procedure was carried out for seven days starting from the day following depilation.

### (4) BrdU Administration

24 hours and 48 hours prior to collection of dorsal body hair skin from C57BL/6N mice on which the drug had been applied for 7 days, a syringe (Terumo Corporation; Japan) was used to administer 0.5 mL of 10 mg/mL BrdU/physiological saline solution to the abdomen in the region of the hind legs of each mouse.

### (5) Mouse Skin Tissue Collection and Fixation

Following euthanization by cervical dislocation of the C57BL/6N mice on the eighth day at the same time as when administration had been carried out, skin tissue was collected in a manner such as would not cause damage to the hair bulb. The collected skin tissue was immersed for 16 to 24 hours in SuperFix (Toyobo Co., Ltd.; Japan) fixative solution, following which this was washed four times in 1×PBS, placed in fresh 1×PBS, and stored. The necessary portion of the collected skin tissue was cut out therefrom, and a paraffin fluid exchange apparatus (Leica Microsystems GmbH; Germany) was used to fabricate a paraffin block therefrom.

### (6) Fabrication of Sections

A microtome (Leica Microsystems GmbH; Germany) was used to section the block of paraffinized skin tissue, section were affixed to platinum-coated glass slides (Platinum Pro (Matsunami Glass Ind., Ltd.; Japan)), these were allowed to stand for 8 to 10 hours in warm steam at 40° C., and dried.

### (7) BrdU Immunostaining

To carry out deparaffinization, glass slides on which samples were mounted were immersed in order for 3 minutes each in xylene, xylene, 100% ethanol, 95% ethanol, and 70% ethanol. Deparaffinized glass slides were immersed for 30 minutes in 2M HCl at room temperature. The glass slides were placed in a Venta Discovery Ultra (autostaining apparatus) and the program was launched. Anti-BrdU antibody-Proliferation Marker (abcam; U.K.) primary antibody was diluted 160× in diluent (1% BSA; 0.1% TX100/PBS), 100 μL/number of samples being added, the timing of which coincided with addition of primary antibody. Hoechst (Hoechst 33342) and Donkey Anti-Sheep IgG H&L (Alexa Flour 594) (Thermo Fisher Scientific; USA) secondary antibody was diluted 500× in diluent (1% BSA; 0.1% TX100/PBS), 100 μL/number of samples being added. Following completion of program, these were cleaned by washing in 0.1% TX100/PBS, and were immersed in 1×PBS. Following dripping of 80 μL/number of samples of water-soluble mounting medium (0.5% gallic acid; 90% glycerol/PBS), a cover glass was placed thereover, yellow chips were used to press out air therefrom, excess mounting medium was removed as cover glass position was corrected using an aspirator, and colorless nail polish (for manicure use) was used to seal the four sides of the cover glass. Determination was made that the colorless nail polish had dried, images were scanned using an Axio Scan, and analysis was carried out.

### (8) BrdU Measurement Method

A 5000-pm line was drawn, epidermal layer, dermal layer, and hair follicle BrdU counts within the region bounded thereby being measured.

### 2. Results

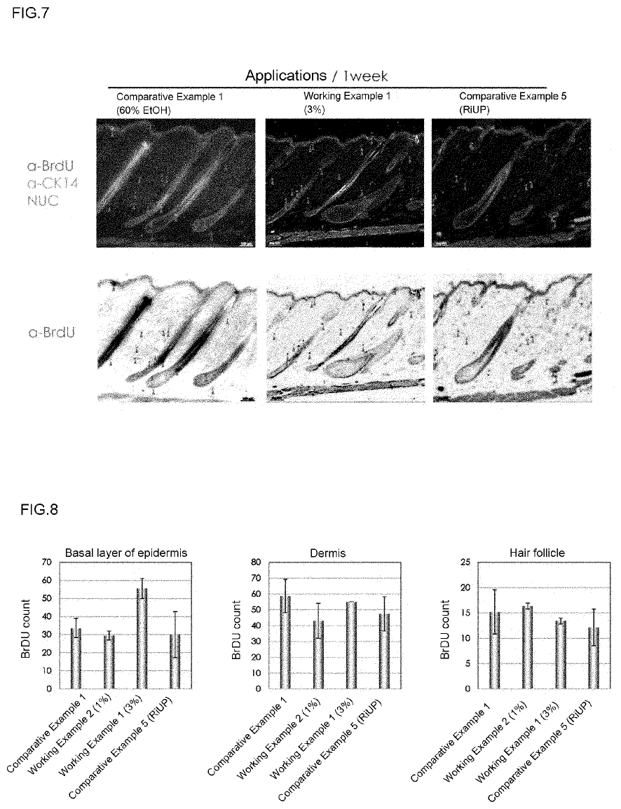

At the present exemplary test, depilated C57BL/6N mice were used, application of a drug solution for testing in the form of palmitoyl dipeptide-5 diaminobutyloyl hydroxythreonine and palmitoyl dipeptide-5 diaminohydroxybutyrate that had been dissolved in 60% ethanol to obtain concentrations of 1% and 3% being carried out in consecutive daily fashion. Application of a negative control in the form of 60% ethanol and of a positive control in the form of RiUP (5% formulation) was carried out. Application thereof was carried out once per day for 7 days so as to achieve a total applied amount of 100 μL. Furthermore, at the same time on the sixth and seventh days, BrdU was administered via the abdomen, skin tissue being collected on the following day. The collected skin tissue was paraffinized and sections were fabricated therefrom, and BrdU immunostaining was carried out, observation of cellular activity being carried out as a result of measurement of BrdU counts. Results are shown in FIG. 7 and FIG. 8.

As is clear from FIG. 7 and FIG. 8, as compared with the negative control and the positive control, application of palmitoyl dipeptide-5 diaminobutyloyl hydroxythreonine and palmitoyl dipeptide-5 diaminohydroxybutyrate, which are the active ingredients of a means in accordance with the present invention, permitted attainment of a trend toward increased mitogenic activity, mitogenic activity being improved at least at the hair follicle for 3% palmitoyl dipeptide-5 diaminobutyloyl hydroxythreonine and palmitoyl dipeptide-5 diaminohydroxybutyrate, permitting attainment of results which suggested that the active ingredients of a means in accordance with the present invention possesses hair growth activity. Furthermore, it was also determined that application of palmitoyl dipeptide-5 diaminobutyloyl hydroxythreonine and palmitoyl dipeptide-5 diaminohydroxybutyrate, which are the active ingredients of a means in accordance with the present invention, caused increase in mitogenic activity at the basal layer of the epidermis, and it was determined that the active ingredients of a means in accordance with the present invention permitted attainment of scalp care effect. It is clear that palmitoyl dipeptide-5 diaminobutyloyl hydroxythreonine and palmitoyl dipeptide-5 diaminohydroxybutyrate, which are the active ingredients of a means in accordance with the present invention, exhibit excellent hair growth activity, and exhibit excellent effect such as will also simultaneously permit achievement of scalp care effect at locations where applied.

### Exemplary Test 3: Evaluation of Hair-Diameter-Increasing Activity Caused by a Mixture of Palmitoyl Dipeptide-5 Diaminobutyloyl Hydroxythreonine and Palmitoyl Dipeptide-5 Diaminohydroxybutyrate

### 1. Materials and Methods

### (1) Experimental Animals

Dorsal body hair skin was collected from C57BL/6N mice (SLC) of age 7 to 8 weeks in accordance with a procedure similar to that at Exemplary Test 1, above, the drug being applied after the hair groups derived from the dorsal body hair skin that had been fabricated were transplanted in Balb/c nu/nu mice (SLC) of age 4 to 6 weeks. Testing and breeding of animals complied with pertinent laws, regulations, ordinances, and guidelines, and was performed with the approval of the Experimental Ethics Review Board of the Institute of Physical and Chemical Research.

### (2) Drugs

The following drugs were used for testing.

Working Example 1: 3% solution of mixture of palmitoyl dipeptide-5 diaminobutyloyl hydroxythreonine and palmitoyl dipeptide-5 diaminohydroxybutyrate
Working Example 2: 1% solution of mixture of palmitoyl dipeptide-5 diaminobutyloyl hydroxythreonine and palmitoyl dipeptide-5 diaminohydroxybutyrate
Comparative Example 1: 60% aqueous ethanol solution
Comparative Example 5: RiUP X5 Plus (registered trademark) (Taisho Pharmaceutical Co., Ltd.; Japan)

### (3) Method for Measuring Increase in Hair Diameter

Measurement of increase in hair diameter was carried out using hair shafts which had grown following completion of the third cycle at Exemplary Test 1. Of the hair shafts that were collected, two zigzag hairs were used. Three locations were selected in regions where diameter was large in the central portions thereof using a square 100 μm on a side. Measurement of selected regions was carried out at five locations.

### 2. Results

Hairs following application of the drug were used, results of measurement of hair shaft diameter being shown in FIG. 9. Furthermore, results of evaluation of fractional increase from the hair shaft diameters at Comparative Example 1 for which 60% aqueous ethanol solution serving as control was applied are shown in TABLE 7.

TABLE 7

| | Working | Working | Working |
|---|---|---|---|
| Component applied | Example 1 | Example 2 | Example 5 |
| thereto | (3%) | (1%) | (RiUP X5 Plus) |
| Percent increase (%) | 115 | 110 | 115 |

It was determined that application of palmitoyl dipeptide-5 diaminobutyloyl hydroxythreonine and palmitoyl dipeptide-5 diaminohydroxybutyrate, which are the active ingredients of a means in accordance with the present invention, caused occurrence of a clear increase in hair diameter as compared with Comparative Example 1 at which 60% aqueous ethanol solution serving as control was applied. The degree to which this increase in hair diameter occurred was equivalent to that at Comparative Example 5, the activity thereof being found to be equivalent to that of the commercially available hair growth agent RiUP X5 Plus.

### Exemplary Test 4: Evaluation of Human Dermal Papilla Cell FGF-7 Gene and VEGF Gene Expression

### 1. Materials and Methods

### (1) Human Dermal Papilla Cells and Culture Medium

Human dermal papilla cells (Catalog No. CA60205a; Caucasian; derived from 29-year-old male; Toyobo Co., Ltd. (Japan)) were purchased, testing and evaluation being carried out with maintenance and culture of cells being performed as described in the protocol.

### (2) Drugs

As drugs for testing, drug solutions of the following respective concentrations (final concentrations) were prepared and used.

Working Example 1: 3% solution of mixture of palmitoyl dipeptide-5 diaminobutyloyl hydroxythreonine and palmitoyl dipeptide-5 diaminohydroxybutyrate
Working Example 3: 0.1% solution of mixture of palmitoyl dipeptide-5 diaminobutyloyl hydroxythreonine and palmitoyl dipeptide-5 diaminohydroxybutyrate
Working Example 4: 0.025% solution of mixture of palmitoyl dipeptide-5 diaminobutyloyl hydroxythreonine and palmitoyl dipeptide-5 diaminohydroxybutyrate
30 μM minoxidil
100 μM adenosine

### (3) Test Procedure

A 96-well plate was seeded with human dermal papilla cells so as to obtain 5×10⁴ thereof per well. Following culture for 1 day within a CO₂ incubator (5% CO₂; 37° C.), the culture medium was replaced with culture medium which contained the respective drugs for testing. The cell plate was thereafter returned to the CO₂ incubator, and this was further cultured for 24 hours or 72 hours. Following culture, total RNA was extracted from the respective wells and was recovered, and this was reverse-transcribed into cDNA. The cDNA that was prepared was used to measure FGF-7 gene expression in accordance with the real-time PCR method. The GAPDH gene was used as an internal standard, the amount of FGF-7 gene expression being calculated relative to the negative control group.

A FastGene RNA Basic Kit (Catalog No. FG-80250; Nippon Genetics Co., Ltd. (Japan)) was used to recover total RNA from cells.

100 μL of lysis buffer RL was added thereto per well, and the cells were lysed by pipetting. 100 μL of 70% ethanol was added to the cell lysate, and this was mixed by pipetting. The sample solution was added to a FastGene RNA binding column, and this was centrifuged at room temperature for 1 minute at 10000 g. The filtrate that passed through the column was discarded from the collection tube, and after returning the FastGene RNA binding column to its original collection tube, 600 μL of wash buffer RW1 was added to the FastGene RNA binding column, and this was centrifuged at room temperature for 1 minute at 10000 g. The FastGene RNA binding column was transferred to a new collection tube that was placed thereat, 700 μL of wash buffer RW2 was added to the FastGene RNA binding column, and this was centrifuged at room temperature for 1 minute at 10000 g. The FastGene RNA binding column was transferred to a new collection tube that was placed thereat, and this was centrifuged at room temperature for 1 minute at 15000 g. The FastGene RNA binding column was transferred to a new collection tube that was placed thereat, 50 μL of elution buffer RE was added at the center of the membrane of the FastGene RNA binding column, and this was centrifuged at room temperature for 1 minute at 10000 g to recover the purified RNA. Concentration of the recovered RNA was measured using a NanoDrop Lite (Catalog No. ND-LITE; Thermo Fisher Scientific K.K.), and this was stored at −80° C. until the following cDNA creation procedure.

A FastGene scriptase II cDNA synthesis 5× Ready Mix (Catalog No. NE-LS64; Nippon Genetics Co., Ltd. (Japan)) was used to synthesize cDNA. Dilution with RNase Free Water was carried out so as to cause concentration of total RNA produced in a new tube to be 20 ng/mL, 4 μL of FastGene scriptase II cDNA synthesis 5× Ready Mix was added to 16 μL of this sample solution, and this was agitated by vortexing. A MiniAmp thermal cycler (Thermo Fisher Scientific K.K.) was used to incubate this at 25° C. for 10 minutes, 42° C. for 60 minutes, and 85° C. for 5 minutes to synthesize cDNA.

The cDNA that was synthesized in accordance with the foregoing method was used to carry out real-time PCR. At prescribed wells in a 96-well plate, respective dilute solutions of cDNA template were added, Thunderbird SYBR qPCR Mix (Catalog No. QPS-201; Toyobo Co., Ltd. (Japan)) and primer were added thereto and mixed therewith, and gene expression was analyzed using a QuantStudio 7 Flex Real-Time PCR System (Catalog No. 4485693; Thermo Fisher Scientific K.K.). The PCR reaction was such that 40 cycles of 95° C. for 5 seconds, 60° C. for 30 seconds, 72° C. for 30 seconds were carried out.

Primers specific for the VEGF gene, primers specific for the FGF-7 gene, and primers specific for the GAPDH gene which was used as internal standard, these having been used for testing, are indicated below.

Primers for detecting FGF-7 gene expression
Forward:
(Sequence No. 1)
tctgtcgaacacagtggtacctgag
Reverse:
(Sequence No. 2)
gccactgtcctgatttccatga
Primers for detecting VEGF gene expression
Forward:
(Sequence No. 3)
atcttcaagccatcctgtgtgc
Reverse:
(Sequence No. 4)
caaggcccacagggattttc
Primers for detecting GAPDH gene expression
Forward:
(Sequence No. 5)
catccctgcctctactggcgctgcc
Reverse:
(Sequence No. 6)
ccaggatgcccttgagggggccctc

Relative amounts of expression of the respective genes was calculated as follows.

For each gene, Ct value (number of PCR cycles) was calculated based on the intersection of the amplification curve with the threshold line. The relative amount of expression is the target gene Ct value less the internal standard GAPDH gene Ct value.

### 2. Results

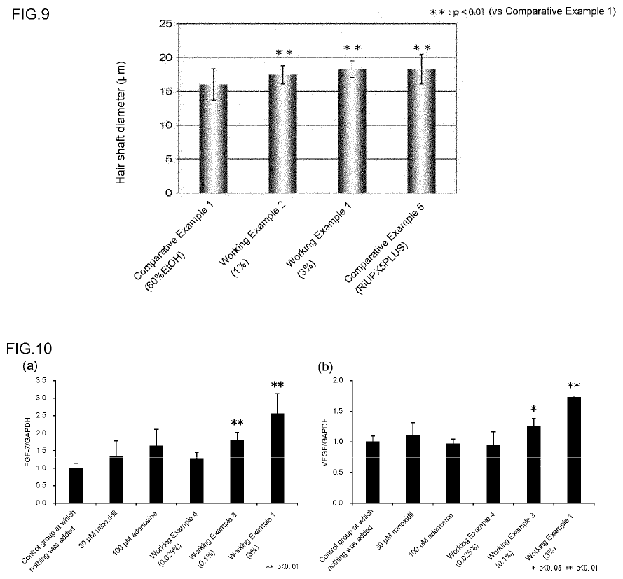

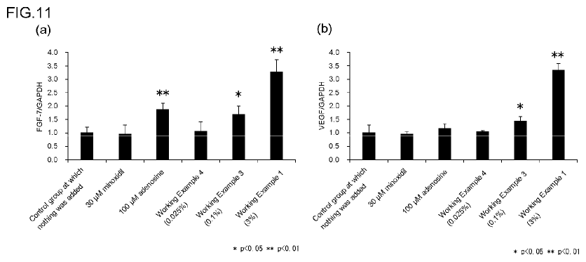

Change in amount of expression of the FGF-7 gene and the VEGF gene following action for 24 hours and 72 hours by mixtures of palmitoyl dipeptide-5 diaminobutyloyl hydroxythreonine and palmitoyl dipeptide-5 diaminohydroxybutyrate on human dermal papilla cells was measured, the results thereof being respectively shown in FIG. 10 (24 hours) and FIG. 11 (72 hours).

As shown in FIG. 10, at the group at which a mixture of palmitoyl dipeptide-5 diaminobutyloyl hydroxythreonine and palmitoyl dipeptide-5 diaminohydroxybutyrate at a final concentration of 3% was allowed to act for 24 hours on human dermal papilla cells, it was found that the amount of expression of the FGF-7 gene and the amount of expression of the VEGF gene had increased significantly as compared with the control group at which nothing was added.

In addition, this amount of expression of the FGF-7 gene and this amount of expression of the VEGF gene were each greater than that which was produced as a result of action of either minoxidil or adenosine.

Furthermore, at the group at which a mixture of palmitoyl dipeptide-5 diaminobutyloyl hydroxythreonine and palmitoyl dipeptide-5 diaminohydroxybutyrate at a final concentration of 0.1% was allowed to act for 24 hours on human dermal papilla cells, it was also found that the amount of expression of the FGF-7 gene and the amount of expression of the VEGF gene had significantly increased as compared with the control group at which nothing was added.

Also, as shown in FIG. 11, at the group at which a mixture of palmitoyl dipeptide-5 diaminobutyloyl hydroxythreonine and palmitoyl dipeptide-5 diaminohydroxybutyrate at a final concentration of 3% was allowed to act for 72 hours on human dermal papilla cells, it was found that the amount of expression of the FGF-7 gene and the amount of expression of the VEGF gene had significantly increased as compared with the control group at which nothing was added. Furthermore, it was found that the extent to which these amounts of genetic expression had increased was greater than that which was produced as a result of action thereof for 24 hours.

In addition, this amount of expression of the FGF-7 gene and this amount of expression of the VEGF gene were each greater than that which was produced as a result of action of either minoxidil or adenosine.

Furthermore, at the group at which a mixture of palmitoyl dipeptide-5 diaminobutyloyl hydroxythreonine and palmitoyl dipeptide-5 diaminohydroxybutyrate at a final concentration of 0.1% was allowed to act for 72 hours on human dermal papilla cells, it was also found that the amount of expression of the FGF-7 gene and the amount of expression of the VEGF gene had significantly increased as compared with the control group at which nothing was added.

It is known that, in human dermal papilla cells, increased expression of the FGF-7 gene and increased expression of the VEGF gene each contributes to increase in hair growth activity in humans. Based on the results of testing indicated above, it is indicated that palmitoyl dipeptide-5 diaminobutyloyl hydroxythreonine and palmitoyl dipeptide-5 diaminohydroxybutyrate cause increase in the amounts of expression of both the FGF-7 gene and the VEGF gene in human dermal papilla cells, and that causing these to act for a longer period of time thereon will produce a greater increase in the amounts of expression of these genes. It is thus clear that palmitoyl dipeptide-5 diaminobutyloyl hydroxythreonine and palmitoyl dipeptide-5 diaminohydroxybutyrate will be very useful as active ingredients for causing increase in hair growth activity in humans.

### INDUSTRIAL UTILITY

As a result using palmitoyl dipeptide-5 diaminobutyloyl hydroxythreonine and palmitoyl dipeptide-5 diaminohydroxybutyrate as active ingredients in a hair growth agent which is a topical agent, a means in accordance with the present invention makes it possible to provide a novel scalp care agent and hair growth agent that exhibit scalp care effect as well as effect in terms of causing increase in hair shaft diameter and effect in terms of improving maximum hair shaft length and effect in terms of improving hair shaft elongation rate and hair shaft growth promotion effect at head hair and at hair of the eyelashes and/or eyebrows and/or the like.

1. A hair growth agent which is a topical agent that contains palmitoyl dipeptide-5 diaminobutyloyl hydroxythreonine and palmitoyl dipeptide-5 diaminohydroxybutyrate.

2. The hair growth agent according to claim 1 wherein the palmitoyl dipeptide-5 diaminobutyloyl hydroxythreonine and the palmitoyl dipeptide-5 diaminohydroxybutyrate are present therein in an amount that is 0.001 wt % to 20 wt % of the entirety.

3. The hair growth agent according to claim 1 wherein the palmitoyl dipeptide-5 diaminobutyloyl hydroxythreonine and the palmitoyl dipeptide-5 diaminohydroxybutyrate are present therein in an amount that is 0.005 wt % to 10 wt % of the entirety.

4. The hair growth agent according to claim 1 wherein the palmitoyl dipeptide-5 diaminobutyloyl hydroxythreonine and the palmitoyl dipeptide-5 diaminohydroxybutyrate are present therein such that a ratio thereof when expressed as a ratio of percents by mass (the palmitoyl dipeptide-5 diaminobutyloyl hydroxythreonine/the palmitoyl dipeptide-5 diaminohydroxybutyrate) is 99/1 to 1/99.

5. The hair growth agent according to claim 1 for use in causing new hair growth or hair shaft growth promotion.

6. The hair growth agent according to claim 1 used for causing improvement in hair shaft elongation rate.

7. The hair growth agent according to claim 1 used for causing improvement in maximum hair shaft length.

8. The hair growth agent according to claim 1 used for causing increase in hair shaft diameter.

9. The hair growth agent according to claim 1 used for causing increase in number of hairs.

10. The hair growth agent according to claim 1 in liquid solution form.

11. The hair growth agent according to claim 1 for use on head hair, eyelashes, and/or eyebrows.

12. A hair growth method comprising administering the hair growth agent according to claim 1 to a subject.
